(19) United States
(12) Patent Application Publication
(10) Pub. No.: US 2025/0241964 A1
(43) Pub. Date: Jul. 31, 2025

(54) SCALABLE METHOD FOR PRODUCING RETINAL PIGMENT EPITHELIUM (RPE) CELLS

(71) Applicant: Eyestem Research Private Limited, Karnataka Bengaluru (IN)

(72) Inventors: Rajarshi PAL, Karnataka Bengaluru (IN); Harshini SURENDRAN, Tamilnadu Coimbatore (IN); Lalitha SOUNDARARAJAN, Karnataka Bangalore (IN); Janavi SUBRAMANI, Karnataka Bangalore (IN)

(21) Appl. No.: 18/853,249

(22) PCT Filed: Oct. 13, 2023
(86) PCT No.: PCT/IN2023/050938

§ 371 Date: Oct. 1, 2024

(30) Foreign Application Priority Data
Oct. 14, 2022 (IN) 202241058893

(51) Int. Cl. A61K 35/30; C12N 5/079

(52) U.S. Cl. CPC A61K 35/30; C12N 5/0621; C12N 2506/45; C12N 2509/00; C12N 2533/90

(57) ABSTRACT

The present disclosure provides a method for obtaining RPE (retinal pigment epithelium) cells from iPSCs (induced pluripotent stem cells). The method involves: (a) generating embryoid bodies from a culture of iPSCs, in which the embryoid bodies are in non-adherent suspension culture, (b) plating the embryoid bodies on a culture dish coated with a suitable extracellular matrix in differentiation induction media (DIM), in which the DIM contains at least one WNT pathway inhibitor and at least two SMAD pathway inhibitors, (c) culturing the neuroectoderm lineage in differentiation propagation media (DPM) for rosette formation, (d) culturing the rosettes of step (c) in Retinal Pigment Epithelium Maturation Media (RPEMM) to facilitate retinal progenitor cells formation, and (e) plating the retinal progenitor cells of step (d) on a culture dish coated with suitable extracellular matrix in RPEMM to obtain RPE cells.

### FIELD OF INVENTION

The present disclosure relates to the field of cell culture techniques and cell differentiation, and methods thereof, and particularly to a cell culture method for producing retinal pigment epithelium (RPE) cells from induced pluripotent stem cells (iPSCs).

### BACKGROUND OF THE INVENTION

Macular degeneration also known as age-related macular degeneration (AMD) is a foremost cause of blindness in people over the age of 60, worldwide. Clinically, this disease starts with distortion in central vision, which is caused due to a damage in the macula, and eventually leads to legal blindness. Vision loss has a significant impact on quality of life and incurs a substantial cost to the economy. Furthermore, AMD is a complex and progressive neurodegenerative disorder that triggers visual impairment due to the loss of retinal pigmented epithelium (RPE) and the light-sensitive photoreceptors, which they support, protect. and provide nutrition. Currently, there is no curative treatment for the most common form of this disease, i.e., dry AMD. Therefore, there is an immediate need to develop therapeutic strategies to address the loss of RPE cells and provide a cure for AMD.

### SUMMARY OF THE INVENTION

In an aspect of the present disclosure, there is provided a method for obtaining RPE (retinal pigment epithelium) cells from iPSCs (induced pluripotent stem cells) comprising the steps of: (a) generating embryoid bodies from a culture of iPSCs, wherein the embryoid bodies are in non-adherent suspension culture, (b) plating the embryoid bodies on a culture dish coated with a suitable extracellular matrix in differentiation induction media (DIM) and culturing for 6-8 days to obtain neuroectoderm lineage, wherein the DIM comprises at least one WNT pathway inhibitor and at least two SMAD pathway inhibitors, (c) culturing the neuroectoderm lineage for 11-22 days in differentiation propagation media (DPM) for rosette formation, wherein the DPM does not comprise any inhibitor, (d) culturing the rosettes of step (c) in Retinal Pigment Epithelium Maturation Media (RPEMM) for 23-45 days to facilitate retinal progenitor cells formation, and (e) plating the retinal progenitor cells of step (d) on a culture dish coated with suitable extracellular matrix and culturing for 47-75 days in RPEMM to obtain RPE cells.

In an aspect of the present disclosure, there is provided a retinal pigment epithelium cell, or a population thereof, produced by the method as disclosed herein.

In an aspect of the present disclosure, there is provided a pharmaceutical composition comprising the retinal pigment epithelium cells produced by the method as disclosed herein; and pharmaceutically acceptable carriers.

In an aspect of the present disclosure, there is provided a method of treating a retinal degeneration disease in a subject comprising: administering the composition as disclosed herein to the subject.

These and other features, aspects, and advantages of the present subject matter will be better understood with reference to the following description. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the Differentiation of RPE, A) Embryoid body formation from iPSCs and their specification towards retinal fate through neuroectoderm induction: B) Formation of rosette-like structures and epithelial clusters indicating the onset of retinal differentiation: C) Retinal progenitors showing fate commitment towards RPE cells with the onset of pigmentation: D) RPE cultures with increase in pigmentation levels and typical hexagonal morphology: E) RPE culture plate with visible (by naked eye) patches of pigmentation: F) Mature RPE cell pellet after 70-80 days in culture: Scale bars 100 μm, in accordance with an embodiment of the present disclosure.

FIG. 2 depicts the Characterization of RPE by immunofluorescence using stage-specific antibodies, A-D) Immunostaining of iPSC derived RPE cultures to check for marker expression during progressive days in retinal differentiation. Images showing the expression of RPE commitment marker Microphthalmia-associated transcription factor (MITF) (A), pigmentation specific proteins, Tyrosinase related protein (TYRP1) (B) and melanocyte protein (PMEL17) (C), RPE maturation marker, (RPE65) (D): Quantification of the purity RPE differentiation by immunophenotyping, Flow cytometry shows high percentage of late stage RPE maturation markers like TYRP1, RPE65 and TYROSINASE-TYR (E), in accordance with an embodiment of the present disclosure.

FIG. 3 depicts the Gene expression analysis using qPCR for selected markers represented as fold change compared to iPSC, All the positive markers (A) have higher expression and the negative markers (B) show a negligible expression in iPSC derived RPE: C) ELISA based quantification of secreted Pigment Epithelium Derived growth factor (PEDF) from the in vitro culture supernatant at two different time points (day 75 and day 120): Images and graphs are representative of minimum three independent experiments, in accordance with an embodiment of the present disclosure.

FIG. 4 depicts A) Phase contrast microscopic images of committed and mature RPE cells under low (10X) and high magnification (20×) showing heavy pigmentation: B) Pictures of the 6 well-plate with RPE cultures at late stage and 15 ml tubes containing RPE cells after centrifugation (inset) during the process of enrichment and scale-up: C) and D) Gene expression profiles showing the purity of RPE cells post enrichment when compared with non-enriched population-Representative heat maps comprising of important non-RPE gene sets showing down regulation in enriched RPE and representative heat maps comprising of important RPE-specific gene sets showing upregulation in enriched RPE, in accordance with an embodiment of the present disclosure.

FIG. 5 depicts A) Behavioural analysis (functional test) results in balanced salt solution (BSS, vehicle) and RPE injected RCS rats by measuring Optokinetic Threshold (OKT): B) Quantification of retinal thickness and number of cones from low, middle and high dose RPE transplanted animals between nasal and temporal regions of the retina was calculated by immunostaining retinal sections with cone-arrestin.; C) Fundus imaging of the saline (BSS) and RPE injected (sub-retinal) eye of RCS rats: D) Immunostaining (HNM) shows survival of transplanted RPE cells in the subretinal space at P90 with preserved ONL layer (cone-arrestin) and a good looking retina indicating vision rescue, in accordance with an embodiment of the present disclosure.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “Induced pluripotent cells (iPSCs)” as used herein refers to pluripotent cells that are derived from adult somatic cells upon ectopic expression of a set of transcription factors. The developmental stages that iPSCs go through are the formation of neural ectoderm, eye field specification, and bilayered optic cup from the optic vesicle.

The term “retinal pigment epithelium (RPE) cells” as used herein refers to a monolayer of pigmented cells derived from the neuroectodermal layer of the optic cup and constitutes the outermost layer of retina. The RPE are needed to keep the retina alive and enable photoreceptor cells to detect light.

The term “Embryoid bodies (EB)” as used herein refers to the three-dimensional aggregates formed in suspension by pluripotent stem cells (PSC), including embryonic stem cells (ESC) and induced pluripotent stem cells (iPSC), that mimic the structure of the developing embryo and have the potential to develop into cells of all three germ layers-ectoderm, mesoderm, and endoderm. EB differentiation is a common platform to generate specific cell lineages from PSCs.

The term “rosettes” as used herein refers to the developmental signature of neuroprogenitors in cultures of differentiating embryonic stem cells: rosettes are radial arrangements of columnar cells that expresses early neuroectodermal markers such as Pax6 and Sox1 and are capable of differentiating into various region-specific neuronal and glial cell types in response to appropriate developmental cues.

The term “inhibitor” as used herein refers to an agent that blocks or dampens a biochemical or biological response when bound to a receptor or a ligand of the receptor. WNT inhibitors, SMAD inhibitors and ROCK inhibitors are the various inhibitors used as part of the present disclosure. For example, “WNT inhibitor” inhibits WNT signaling by preventing ligand-receptor interactions or WNT receptor maturation.

The term “subject” as used herein refers to both human and veterinary subjects, for example, rats, non-human primates, dogs, cats, horses, rabbits, pigs, mice, and cows.

The term “pharmaceutically acceptable carriers” as used herein refers to those substances that are useful for practicing the methods and forming the compositions disclosed herein. In general, the nature of the carrier will depend on the particular mode of administration being employed. For example, for subretinal delivery carriers, such as Balanced salt solution (BSS) or Hank's balanced salt solution (HBSS).

The term “confluency” as used herein refer to the percentage of the growth medium area (surface area of the culture dish) covered by adherent cells. For example, 60% confluency indicates that 60 out of 100 parts of the growth surface are occupied by cells. Confluency is used as an indicator of cell growth and expansion during cell culture experiments.

The term “gradually” as used herein refers to an action performed slowly or by small degrees over a period of time. According to the present disclosure, the development of embryoid bodies is facilitated by shifting the media in contact with the EBs from growth media to differentiation induction media (DIM) by slowly increasing the proportion of DIM, while decreasing the growth media proportion. Thus, in an aspect of the present disclosure, the EBs are developed in a media composition comprising the expansion media and DIM in a ratio of 3:1 for 24 hours: then in a media composition comprising the expansion media and DIM in a ratio of 1:1 for 24 hours; and finally in 100% DIM for 24 hours.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally equivalent products, compositions, and methods are clearly within the scope of the disclosure, as described herein.

Many ophthalmic diseases, such as age-related macular degeneration, are associated with a degeneration or deterioration of the retina itself or of the RPE. It is imperative to achieve photoreceptor rescue and preservation of visual function by subretinal transplantation of RPE cells. There is a need to find ways to produce RPE cells in a robust and scalable method, such as from human stem cells, that can be used for the treatment of retinal degenerative diseases and injuries.

According to the present disclosure, there is provided a method for the generation of RPE through a combination of chemical and manual selection processes for enrichment of RPE at early and late stages of the differentiation process. In principle, iPSCs are coaxed into neuroectodermal fate by suppressing pluripotency factors like FGF2 using dual SMAD and WNT inhibition. This is achieved by the use of small molecules like SB431542 (4-[4-(1,3-benzodioxol-5-yl)-5-pyridin-2-yl-1H-imidazol-2-yl]benzamide) and LDN193189 (4-(6-(4-(piperazin-1-yl)phenyl) pyrazolo[1,5-a]pyrimidin-3-yl) quinoline).SB431542 inhibited Activin/TGF-β pathways and LDN193189 acted as a BMP4/7 inhibitor while IWR1 (4-(1,3,3a,4,7,7a-Hexahydro-1,3-dioxo-4,7-methano-2H-isoindol-2-yl)-N-8-quinolinyl-Benzamide) silenced the canonical WNT signaling pathway.

To facilitate retinal cell proliferation, the embryoid bodies obtained from iPSCs are cultured in low serum media supplemented with N1, thus forming retinal progenitors which are enriched to produce matured RPE. The matured RPE represent tightly packed hexagonal cells with tight junctions, and brown to black pigmentation with apical-basal polarity. This method generated pigmented and ciliated RPE exhibiting functional characteristics such as polarized secretion of cytokines-PEDF and VEGF. Characterization methods to determine their authenticity and purity are also laid down along with this method. The present disclosure also provides a pharmaceutical composition comprising the RPE cells. Further, the present disclosure provides a method of treating retinal degeneration diseases using the pharmaceutical composition.

In an embodiment of the present disclosure, there is provided a method for obtaining RPE (retinal pigment epithelium) cells from iPSCs (induced pluripotent stem cells) comprising the steps of: (a) generating embryoid bodies from a culture of iPSCs, wherein the embryoid bodies are in non-adherent suspension culture, (b) plating the embryoid bodies on a culture dish coated with a suitable extracellular matrix in differentiation induction media (DIM) and culturing for 6-8 days to obtain neuroectoderm lineage, wherein the DIM comprises at least one WNT pathway inhibitor and at least two SMAD pathway inhibitors, (c) culturing the neuroectoderm lineage for 11-22 days in differentiation propagation media (DPM) for rosette formation, wherein the DPM does not comprise any inhibitor, (d) culturing the rosettes of step (c) in Retinal Pigment Epithelium Maturation Media (RPEMM) for 23-45 days to facilitate retinal progenitor cells formation, and (e) plating the retinal progenitor cells of step (d) on a culture dish coated with suitable extracellular matrix and culturing for 47-75 days in RPEMM to obtain RPE cells.

In an embodiment of the present disclosure, there is provided a method for obtaining RPE (retinal pigment epithelium) cells from iPSCs (induced pluripotent stem cells), wherein the method further comprises enriching the RPE cells, comprising: (a) enzymatically dissociating the RPE cells from the extracellular matrix to obtain a single cell suspension of RPEs: (b) plating the single cell suspension of RPEs of step (a) on a culture dish coated with suitable extracellular matrix and culturing for 75-100 days in RPEMM.

In an embodiment of the present disclosure, there is provided a method for obtaining RPE (retinal pigment epithelium) cells from iPSCs (induced pluripotent stem cells) comprising the steps of: (a) generating embryoid bodies from a culture of iPSCs, wherein the embryoid bodies are in non-adherent suspension culture, (b) plating the embryoid bodies on a tissue culture dish coated with a suitable extracellular matrix in differentiation induction media (DIM) and culturing for 6-8 days to obtain neuroectoderm lineage, wherein the DIM comprises at least one WNT pathway inhibitor and at least two SMAD pathway inhibitors, (c) culturing the neuroectoderm lineage for 11-22 days in differentiation propagation media (DPM) for rosette formation, wherein the DPM does not comprise any inhibitor, (d) culturing the rosettes of step (c) in Retinal Pigment Epithelium Maturation Media (RPEMM) for 23-45 days to facilitate retinal progenitor cells formation, and (e) plating the retinal progenitor cells of step (d) on a culture dish coated with suitable extracellular matrix and culturing for 47-75 days in RPEMM to obtain RPE cells: wherein the method further comprises enriching the RPE cells, comprising: (a) enzymatically dissociating the RPE cells from the extracellular matrix to obtain a single cell suspension of RPEs: (b) plating the single cell suspension of RPEs of step (a) on a culture dish coated with suitable extracellular matrix and culturing for 75-100 days in RPEMM.

In an embodiment of the present disclosure, there is provided a method for obtaining RPE (retinal pigment epithelium) cells from iPSCs (induced pluripotent stem cells), wherein the culture of iPSCs has a confluency in the range of 80%-90%. In another embodiment, wherein the confluent culture of iPSCs have a confluency in the range of 80%-85%.

In an embodiment of the present disclosure, there is provided a method for obtaining RPE (retinal pigment epithelium) cells from iPSCs (induced pluripotent stem cells), wherein generating the embryoid bodies in step (a) comprises: (a) culturing the confluent iPSCs for 24 hours in a growth media to form embryoid bodies, wherein the growth media comprises a ROCK inhibitor, and (b) gradually contacting the developing embryoid bodies from the growth media to the DIM by (i) culturing the developing embryoid bodies in a media composition comprising an expansion medium and DIM in a ratio of 3:1 for 24 hours, (ii) culturing the developing embryoid bodies obtained in step (i) in a media composition comprising the expansion medium and DIM in a ratio of 1:1 for 24 hours, and (iii) culturing the embryoid bodies obtained in step (ii) in DIM for 24 hours followed by plating the embryoid bodies on a tissue culture dish.

In an embodiment of the present disclosure, there is provided a method for obtaining RPE (retinal pigment epithelium) cells from iPSCs (induced pluripotent stem cells), wherein the rosette formation in step (c) comprises: i) maintaining the neuroectoderm lineage in DIM for 24 hours: ii) maintaining the neuroectoderm lineage obtained in step (i) in a media composition comprising DIM and DPM in a ratio of 1:1 for 24-48 hours; and iii) culturing the neuroectoderm lineage obtained from step (ii) in DPM for 24 hours for facilitating rosette formation.

In an embodiment of the present disclosure, there is provided a method for obtaining RPE (retinal pigment epithelium) cells from iPSCs (induced pluripotent stem cells), wherein the retinal progenitor cells formation in step (d) comprises: (i) maintaining the rosettes in the DPM for 24 hours: (ii) maintaining the rosettes obtained in step (i) in a media composition comprising DPM and RPEMM in a ratio of 1:1 for 24 hours; and (iii) culturing the rosettes obtained from step (ii) in RPEMM for 24 hours for facilitating the formation of retinal progenitor cells.

In an embodiment of the present disclosure, there is provided a method for obtaining RPE (retinal pigment epithelium) cells from iPSCs (induced pluripotent stem cells) comprising the steps of: (a) generating embryoid bodies from a culture of iPSCs having a confluency in the range of 80%-90%, wherein the embryoid bodies are in non-adherent suspension culture, (b) plating the embryoid bodies on a culture dish coated with a suitable extracellular matrix in differentiation induction media (DIM) and culturing for 6-8 days to obtain neuroectoderm lineage, wherein the DIM comprises at least one WNT pathway inhibitor and at least two SMAD pathway inhibitors, (c) culturing the neuroectoderm lineage for 11-22 days in differentiation propagation media (DPM) for rosette formation, wherein the DPM does not comprise any inhibitor, (d) culturing the rosettes of step (c) in Retinal Pigment Epithelium Maturation Media (RPEMM) for 23-45 days to facilitate retinal progenitor cells formation, and (e) plating the retinal progenitor cells of step (d) on a culture dish coated with suitable extracellular matrix and culturing for 47-75 days in RPEMM to obtain RPE cells: wherein generating the embryoid bodies in step (a) comprises: (a) culturing the confluent iPSCs for 24 hours in a growth media to form embryoid bodies, wherein the growth media comprises an expansion media and a ROCK inhibitor, and (b) gradually contacting the developing embryoid bodies from the growth media to DIM by (i) culturing the developing embryoid bodies in a media composition comprising the expansion medium and DIM in a ratio of 3:1 for 24 hours, (ii) culturing the developing embryoid bodies obtained in step (i) in a media composition comprising the expansion medium and DIM in a ratio of 1:1 for 24 hours, and (iii) culturing the embryoid bodies obtained in step (ii) in DIM for 24 hours followed by plating the embryoid bodies on a culture dish: wherein the rosette formation in step (c) comprises: i) maintaining the neuroectoderm lineage in the DIM for 24 hours: ii) maintaining the neuroectoderm lineage obtained in step (i) in a media composition comprising DIM and DPM in a ratio of 1:1 for 24-48 hours; and iii) culturing the neuroectoderm lineage obtained from step (ii) in DPM for 24 hours for facilitating rosette formation; and wherein the retinal progenitor cells formation in step (d) comprises: (i) maintaining the rosettes in the DPM for 24 hours: (ii) maintaining the rosettes obtained in step (i) in a media composition comprising DPM and RPEMM in a ratio of 1:1 for 24 hours; and (iii) culturing the rosettes obtained from step (ii) in RPEMM for 24 hours for facilitating the formation of retinal progenitor cells.

In an embodiment of the present disclosure, wherein the growth medium comprises an expansion media and a ROCK inhibitor: wherein the ROCK inhibitor is (1R,4r)-4-((R)-1-aminoethyl)-N-(pyridin-4-yl) cyclohexanecarboxamide.

In an embodiment of the present disclosure, there is provided a method for obtaining RPE (retinal pigment epithelium) cells from iPSCs (induced pluripotent stem cells), wherein the at least one suitable extracellular matrix is selected from matrigel, laminin, vitronectin, fibronectin, collagen, poly-L-lysine, poly-L-ornithine, or combinations thereof.

In an embodiment of the present disclosure, there is provided a method for obtaining RPE (retinal pigment epithelium) cells from iPSCs (induced pluripotent stem cells), wherein the at least one WNT pathway inhibitor is selected from 4-(1,3,3a,4,7,7a-Hexahydro-1,3-dioxo-4,7-methano-2H-isoindol-2-yl)-N-8-quinolinyl-Benzamide, 5-(Phenylsulfonyl)-N-piperidin-4-yl-2 (trifluoromethyl)benzenesulfonamide, 2-(2′,3-Dimethyl-[2,4′-bipyridin]-5-yl)-N-(5-(pyrazin-2-yl) pyridin-2-yl) acetamide, 2-(4-(2-methylpyridin-4-yl)phenyl)-N-(4-(pyridin-3-yl)phenyl) acetamide, 8-Tetrahydro-2-[4-(trifluoromethyl)phenyl]-4H-thiopyrano[4,3-d]pyrimidin-4-one, or combinations thereof.

In an embodiment of the present disclosure, there is provided a method for obtaining RPE (retinal pigment epithelium) cells from iPSCs (induced pluripotent stem cells), wherein the at least two SMAD pathway inhibitors are selected from 4-[4-(1,3-benzodioxol-5-yl)-5-pyridin-2-yl-1H-imidazol-2-yl]benzamide, 4-(6-(4-(piperazin-1-yl)phenyl) pyrazolo[1,5-a]pyrimidin-3-yl) quinoline, 3-[(1R)-1-(2,6-dichloro-3-fluorophenyl) ethoxy]-5-(1-piperidin-4-ylpyrazol-4-yl) pyridin-2-amine, 5-chloro-2-N-[2-methoxy-4-[4-(4-methylpiperazin-1-yl) piperidin-1-yl]phenyl]-4-N-(2-propan-2-ylsulfonylphenyl) pyrimidine-2,4-diamine, 9-ethyl-6,6-dimethyl-8-(4-morpholin-4-ylpiperidin-1-yl)-11-oxo-5H-benzo[b]carbazole-3-carbonitrile, 5-chloro-2-N-(5-methyl-4-piperidin-4-yl-2-propan-2-yloxyphenyl)-4-N-(2-propan-2-ylsulfonylphenyl) pyrimidine-2,4-diamine, or combinations thereof. In another embodiment of the present disclosure, wherein the at least two SMAD pathway inhibitors 4-[4-(1,3-benzodioxol-5-yl)-5-pyridin-2-yl-1H-imidazol-2-yl]benzamide, and 4-(6-(4-(piperazin-1-yl)phenyl) pyrazolo[1,5-a]pyrimidin-3-yl) quinoline.

In an embodiment of the present disclosure, there is provided a method for obtaining RPE (retinal pigment epithelium) cells from iPSCs (induced pluripotent stem cells), wherein the ROCK inhibitor is (1R,4r)-4-((R)-1-aminoethyl)-N-(pyridin-4-yl) cyclohexanecarboxamide.

In an embodiment of the present disclosure, there is provided a method for obtaining RPE (retinal pigment epithelium) cells from iPSCs (induced pluripotent stem cells), wherein the enzymatic dissociation of RPE cells in step (a) is carried out using an enzyme selected from the group consisting of Accutase, Tryple select, TrypLE, Gentle Cell Dissociation Reagent (GCDR), and Dispase.

In an embodiment of the present disclosure there is provided a retinal pigment epithelium cell, or a population thereof, produced by the method as disclosed herein.

In an embodiment of the present disclosure there is provided a pharmaceutical composition comprising the retinal pigment epithelium cell or population thereof, as disclosed herein; and pharmaceutically acceptable carriers.

In an embodiment, the pharmaceutically acceptable carriers are selected from Balanced salt solution (BSS) or Hank's balanced salt solution (HBSS).

In an embodiment of the present disclosure there is provided a pharmaceutical composition as disclosed herein, for use in treatment of retinal degeneration disease.

In an embodiment of the present disclosure there is provided a composition, wherein the retinal degeneration disease is selected from a group consisting of, age-related macular degeneration, and retinal diseases associated with early and late-stage photoreceptor degeneration.

In an embodiment of the present disclosure there is provided a method of treating renal degeneration disease in a subject comprising: administering the pharmaceutical composition as disclosed herein to the subject.

Although the subject matter has been described with reference to specific embodiments, this description is not meant to be constructed in a limiting sense.

Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with working examples, which are intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods and experimental conditions described, as such methods and conditions may apply.

### EXAMPLE 1

### Materials

iPSC lines TC-1133 (RUCDR/NIH) was obtained from Eyestem Biobank and can be found at https://eyestem.com/collaborations/research-alliance/.

All critical reagents used in the present disclosure were procured as mentioned in the key resources table.

iPSCs were procured/thawed and maintained as undifferentiated cultures in mTeSR-based medium (expansion medium). Culture media were prepared according to the recipes mentioned in the method.

The iPSCs maintenance and differentiation media can be used for up to 2 weeks when stored at 4° C. Thus, it is not advisable to prepare all the media at once, rather it is better to prepare it when needed at the specific step of the procedure.

All procedures were performed in a BSL-2 certified laboratory biosafety cabinet with standard aseptic techniques. Cultures were grown and maintained in a humidified incubator at 37° C. with 5% CO2.

Cultures were tested regularly for sterility and karyotyping.

Standard cell culture practices like aseptic handling of cultures, wearing personal protective gear along with appropriate equipments like biosafety cabinets, CO2 incubators, centrifuge, water-bath and microscope were used. Culturing of cells in plates coated with extracellular matrices (ECM) were performed in humidified incubators at 37° C. with 5% CO2 levels. Before differentiation, iPSCs were characterized to check for expression of pluripotent markers, chromosomal aberrations by karyotyping, and sterility tests to determine if the cultures are contamination-free (bacterial and mycoplasma).

### EXAMPLE 2

### General Experimental Preparations

The reproducibility of the RPE differentiation method has been tested in multiple iPSC lines, including TC-1133 (RUCDR/NIH: Baghbaderani. B. A., et al., (2015). cGMP-Manufactured Human Induced Pluripotent Stem Cells Are Available for Pre-clinical and Clinical Applications. Stem cell reports, 5 (4). 647-659). ERPLi001-A, ERPLi002-A, and ERPLi003-A (Konala et al., Derivation of three induced pluripotent stem cell lines under feeder-free culture conditions from peripheral blood mononuclear cells (PBMC) of Indian patients suffering from inherited retinal diseases carrying different mutations. Stem Cell Research. 45. 101757). Therefore, similar differentiation results were expected in other iPSC lines following the detailed method as described herein.

The entire method was performed in a sterile environment, such as a biosafety cabinet. The method includes:

1.Preparation of Cell Therapy Systems-Vitronectin (CTS™-VTN) for iPSC Expansion.

Timing: 1 h
a. CTS-VTN (0.9 mg/ml) was thawed at room temperature (20-25° C.) for 5-10 mins, and then placed on ice.
   CTS-VTN was divided into usage-size aliquots in polypropylene tubes and stored at −60° C. to −80° C.
b. Room temperature storage and/or shaking might result in an appearance of light turbidity, which did not impact product performance.
   For coating the wells of a 6-well plate, 60 μL of CTS-VTN was added into a 15 ml tube containing 6 ml of sterile CTS™ DPBS (1X) (−Ca²⁺/−Mg²⁺) at room temperature.
c. Gently resuspended by pipetting the CTS-VTN solution up and down.
   This resulted in a working concentration of 9 μg/ml (i.e., a 1:100 dilution). Diluted CTS-VTN solution can be stored at 4° C. for not more than 7 days. Then, 1 ml of diluted CTS-VTN solution was added to each well of a 6 well plate. The plate was either swirled and/or rocked to ensure even coating.
d. When used to coat a 6 well plate (10 cm²/well) at 1 ml/well, the final concentration will be 0.9 μg/cm².
e. The coated plates were incubated at 37° C. for 1 hour.

The culture plate can now be used or stored at 2° C. to 8° C., wrapped in laboratory film, for up to one week, while making sure the wells do not dry out. A volume of basal media may be added to the well(s) 1 hour after coating to ensure that the wells do not dry out. If a portion of a well does dry out, this well cannot be used. Prior to use, the culture plate has to be pre-warmed to room temperature (20-25° C.).
f. The CTS™_VTN solution was aspirated and discarded immediately prior to use. It is not necessary to rinse off the culture plate after the removal of CTS™ VTN. Cells can be seeded directly onto the CTS™ VTN-coated culture plates.
   2. iPSC culture and maintenance.

Timing: 6-10 days

CTS-VTN coated plates were prepared before starting the method. If a CTS-VTN coated plate that has been stored at 4° C. is used, then the plate must be allowed to equilibrate to room temperature for 1 hour prior to starting.

One cryovial packed with 1 million cells should be thawed into 1 well of a 6 well plate (10 cm² surface area).
a. Five ml of cold mTeSR plus medium was added to a sterile 15 ml tube. A 1:5 ratio is recommended to effectively dilute the Cryostor CS10 (1 ml of cells and 5 ml of mTeSR plus).
b. Cells were removed from the liquid nitrogen storage tank.
c. The cells were quickly thawed in a 37° C. water bath using a “FIG. 8” motion until a pea-sized ball of ice was seen.

To avoid cell death, the cells should not be thawed completely in the water bath.
d. Using a 2 ml pipette, mTeSR plus was slowly added to the cells drop by drop and collected in a 15 ml tube.
e. The 15 ml tube was capped and gently the tube was inverted 4-5 times to mix the CryoStor CS10 and mTeSR plus.
f. The tube was then centrifuged at 200 g, for 3 mins at 25±5° C.
g. While cells were spinning, CTS-VTN was aspirated from the plate.
h. Gently the supernatant was aspirated from the cell pellet and cells were re-suspended in 2 ml of fresh mTeSR plus containing 10 μM ROCK inhibitor (Y-27632).

The use of ROCK inhibitor Y-27632 increased cell survival and cell health. Y-27632 was reconstituted and stored as per manufacturer's instructions. (https://www.tocris.com/products/y-27632-dihydrochloride_1254).

It was ensured to completely remove DMSO present in storage media by washing due to its known toxicity. The operation was done in a gentle and quick manner.
i. The cells were plated onto the CTS-VTN coated well.
j. The culture plate was placed in the incubator: the plate was gently rocked in a plus direction for homogeneous distribution of the cells.
k. The next day, the media was replaced with 2 ml of fresh mTeSR plus to remove Y-27632.

For media changes, medium was always added/removed carefully close to the wall of the wells to avoid detaching the cells.
l. Colonies were expected to appear within 2-3 days.
m. Medium was changed every day and when the colonies covered 70%-80% of the plate they were ready to be passaged.
   3. iPSC Passaging

Timing: 1 hour
a. CTS-VTN-coated plates were prepared as described previously and mTeSR plus was pre-warmed.
b. The medium was aspirated and the well was washed with (1 ml/well of 6 wells) 1XPBS.
c. ReLeSR™ was added to the culture plate and ReLeSR™ was aspirated within 1 minute, such that colonies were exposed only to a thin film of the liquid.

TABLE 1
Correlation of media volume to surface area of culture plate.

| Culture | Surface | | | | | | Culture | |
|---|---|---|---|---|---|---|---|---|
| vessel | area | | DPBS | | ReLeSR | | medium | |
| 6-well plate | 10 | cm²/well | 2 | ml/well | 1 | ml/well | 2 | ml/well |
| 12-well plate | 4 | cm²/well | 1 | ml/well | 0.5 | ml/well | 1 | ml/well |
| 24-well plate | 2 | cm²/well | 0.5 | ml/well | 0.3 | ml/well | 0.5 | ml/well |
| 35-mm dish | 10 | cm² | 2 | ml | 1 | ml | 2 | ml |
| 60-mm dish | 20 | cm² | 4 | ml | 2 | ml | 4 | ml |
| 100-mm dish | 60 | cm² | 12 | ml | 5 | ml | 12 | ml |

d. The plate was incubated at 37° C. for 4 mins+30 seconds.
e. Gently the plate(s) or well(s) were rinsed with 1 ml of mTeSR plus for complete detachment of cells.
f. The colonies were detached by holding the plate with one hand and using the other hand to firmly tap the side of the plate for approximately 30-60 seconds.
g. The detached cell aggregates were transferred to a 15 ml tube using a 5 ml serological pipette
h. Cell aggregates should be appropriately sized for plating (mean aggregate size of approximately 50-200 μm)
i. The sterile 15 ml or 50 ml serological tube was centrifuged for 3 mins at 200 g by keeping acceleration and deceleration at a maximum of 9 for the centrifuge.
j. Upon completion of centrifugation, the supernatant was removed, the pellet was gently tapped, and appropriate volume of mTeSR plus was added along with 10 μM Y-27632, depending on the split ratio, to a 15 ml or 50 ml tube.
k. The cell colonies were plated onto the CTS-VTN coated dishes.
l. The plate was rocked in a plus direction to ensure even distribution of colonies in the wells.
m. The plate was placed in a 37° C. incubator with 5% CO2. It was ensured to not move the plate for 24 hours.
n. After 24 hours, the plate was observed under the microscope to confirm that the colonies were attached to the plate. The next day, media was replaced with 2 ml of fresh mTeSR plus to remove Y-27632.
o. The medium was changed every day until plate(s) reached 70% confluency in less than 7 days.

Any plate(s) that did not become 70% confluent within 7 days were discarded. Before every passage, the supernatant was pooled in from all plate(s) into a single sterile 15 ml tube and stored at −80° C. until it was tested for sterility.

### 4. Matrigel Aliquoting and Coating

g. Matrigel was aliquoted and stored at −20° C. It was important that any material that would come in contact with the Matrigel be ice-cold. Matrigel will solidify and adhere to any item that is above 10° C. Therefore, it was ensured to avoid multiple freeze/thaw cycles.
h. Before beginning, the frozen serological pipettes, pipet tips, 15 ml tubes and matrigel aliquots were placed on ice in the bio-safety cabinet (BSC). It was ensured to spray the ice bucket down thoroughly with 70% ethanol before placing it in the BSC.
i. The matrigel was diluted at 1:100 in cold DMEM-F12 medium. For example, 100 μL matrigel was added in 10 ml DMEM-F12 media.

It was taken care to keep fingertips above the matrigel level, as the warmth from fingertips would cause the matrigel to solidify. It was ensured to frequently change to a new cold pipet tip.

This 1:100 dilution would make 1% matrigel solution ready to use for coating culture plates.
j. Using the cold serological pipette, 1% matrigel solution was transferred to the culture plate at 1 ml/well of 6 well plate.
k. The plate was swirled and/or rocked to ensure even coating.
l. The plate was then incubated at 37° C. for at least 1 hour.
m. Alternatively, if matrigel coated plates were not used on the same day as coating, plates were wrapped in parafilm, and stored in 4° C. fridge for up to 1 week, while making sure wells do not dry out.

A volume of basal media may be added to the well(s) 1 hour after coating to ensure that the wells do not dry out. If a portion of a well does dry out, then the well cannot be used.

Prior to use, matrigel solution was removed. It was avoided to air dry the matrigel-coated wells before seeding cells.

### EXAMPLE 3

Differentiation of iPSC into Retinal Pigmented Epithelial (RPE) Cells
1. Formation of Embryoid Bodies (EBs) by Forced Aggregation of iPSCs.

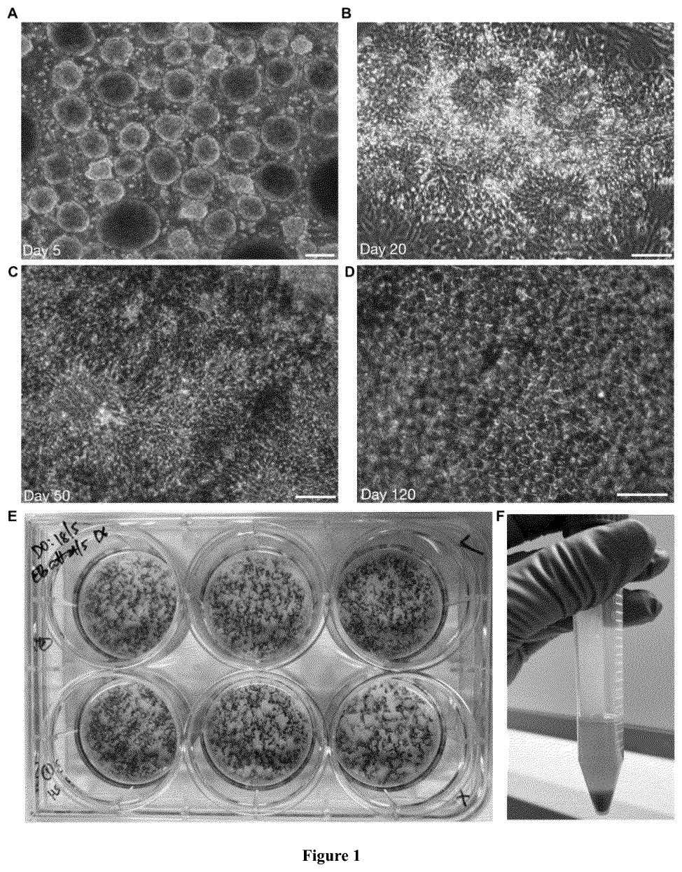

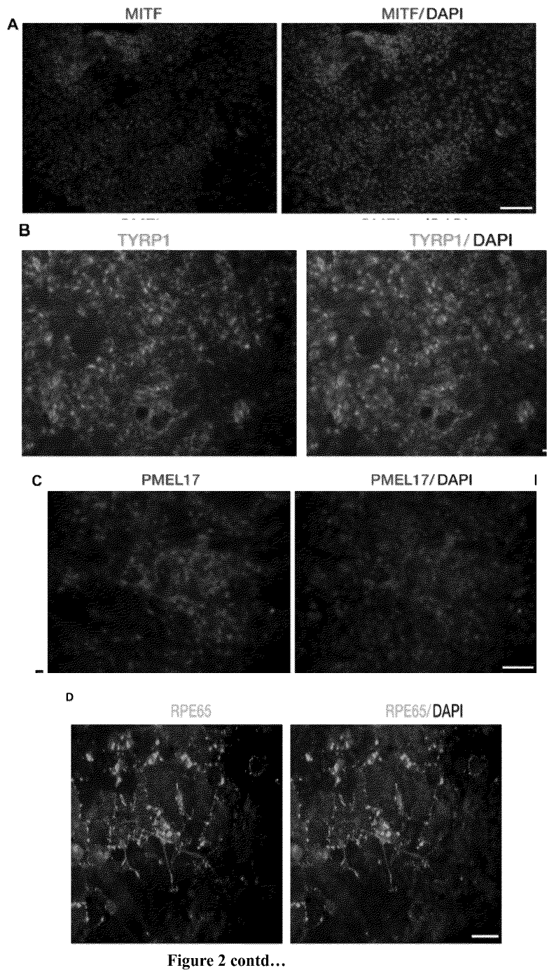

Timing: Day 0-2
a. The mTeSR plus (Expansion medium: cGMP, stabilized feeder-free maintenance medium for iPS cells was procured commercially), CTS™ DPBS (1X) (-Ca²⁺/−Mg²⁺) was prewarmed to 37° C.
   One 6-well plate (at 80% confluency, about 1 million cells per well) was sufficient to initiate the RPE differentiation through EB formation (FIG. 1A).
b. The cells from 6 wells would be going into 6 wells of ultra-low attachment plates (in order to avoid any form of attachment) for the formation of EBs.
c. The ultra-low attachment plates were prepared by adding 1 ml of mTeSR plus medium to each well.
d. The spent medium was aspirated from the plate containing iPSCs and the plate was rinsed once with CTS™ DPBS (1X) (−Ca²⁺/−Mg²⁺) (Refer Table-1 for volume).
e. CTS™ DPBS (1X) (−Ca²⁺/−Mg²⁺) was aspirated and ReLeSR Solution was added to the plate containing iPSCs. The volume of ReLeSR Solution was adjusted for various plate sizes (Refer Table-1 for volume).
f. 1 ml/well of ReLeSR™ was added and ReLeSR™ was aspirated within 1 minute, such that colonies were exposed to a thin film of liquid.
g. The plate was incubated at 37° C. for 4 mins+30 seconds.
h. An appropriate amount of mTeSR plus medium was added to each plate (Refer Table-1 for volume) to stop the dissociation reaction.
i. Gently the dissociated cells were pipetted up and down sufficiently to disperse the colonies into a single-cell suspension.
j. It was made sure to pipet gently to minimize the formation of bubbles.
k. The iPSC suspension was transferred from each well into a separate 15 ml tube and the tube(s) were centrifuged at 200 g for 3 mins to pellet the cells.
l. Carefully the supernatant(s) were aspirated from the iPSC pellet(s).
m. The pellet(s) were resuspended with an appropriate amount (approximately 6 ml) of mTeSR plus medium.
n. The resuspended cells were added to each 6 well ultra-low attachment plate containing 6 ml mTeSR plus medium (1 ml/well) with 10 μM ROCK inhibitor, Y-27632 (growth medium).
o. This day was considered Day 0. The cells were incubated for 24 hours in a 37° C., 5% CO2 incubator to allow them to form EBs. The media was replaced with 2 ml of fresh mTeSR plus (expansion medium) to remove Y-27632.
p. Day 1 and Day 2: Gently the EB plate was swirled and the EBs were brought to the center of the dish. 1 ml of medium as removed from well corner and 1 ml of fresh mTeSR plus medium was added. (FIG. 1A)

### 2. Differentiation Induction and Plating of Embryoid Bodies (EBs) to Obtain Neuroectoderm Lineage

Timing: Day 3-8
a. Differentiation Induction Medium DIM was prepared as per Table-2.

TABLE 2
Differentiation Induction Medium (DIM)

| | For 500 | | Final | |
|---|---|---|---|---|
| Component | ml | | concentration | |
| DMEM/F12 | 425 | ml | | |
| Fetal Bovine Serum (FBS) | 50 | ml | 10% | |
| Sodium pyruvate (100 mM) | 5 | ml | 1 | mM |
| Sodium bicarbonate (7.5% solution) | 5 | ml | 8.92 | mM |
| HEPES buffer (1M) | 5 | ml | 10 | mM |
| Non-essential amino acids (100X) | 5 | ml | 1X | |
| N1 media supplement (100X) | 5 | ml | 1X | |
| IWRI (2 mM) (WNT inhibitor) | 500 | μl | 2 | μM |
| SB431542 (10 mM) (SMAD | 500 | μl | 10 | μM |
| inhibitor) | | | | |
| LDN 193189 (1 mM) (SMAD | 50 | μl | 100 | nM |
| inhibitor) | | | | |
| IGF-1 (100 μg/ml) | 50 | μl | 10 | ng/ml |

The growth factors were reconstituted and stored as per manufacturer's instructions. (https://www.sigmaaldrich.com/IN/en/product/sigma/i0161; https://www.tocris.com/products/sb-431542 1614; https://www.tocris.com/products/ldn-193189-dihydrochloride 6053; https://www.stemcell.com/products/human-recombinant-igf-i.html).
b. Gradually the medium of the suspension cultures with EBs were shifted from mTeSR plus (expansion medium) to differentiation induction media (DIM).
c. Day 3: One part of mTeSR plus media was replaced with DIM (3:1) (Example: For 1 ml media, 750 μl mTeSR plus media, and 250 μl DIM).
d. Day 4: One part of mTeSR plus media was replaced with DIM (1:1) (Example: For 1 ml media, 500 μl mTeSR plus media, and 500 μl DIM).
e. Day 5: Complete media change (100%) was given with DIM.
f. Day 6: Matrigel was coated, and EB were plated.

The EBs were plated on matrigel (extracellular matrix) coated culture dishes. At this point, EBs were expected to be dense, spherical, and have a defined boundary and would easily pool to the center when the plate was swirled.
i. Gently the EB plate was swirled and EBs were transferred from 3 wells of a 6 well plate into a 15 ml tube.
ii. The EBs were allowed to settle to the bottom of the tube at room temperature (20-25° C.) inside the bio-safety cabinet (it would take about 3 min). It was ensured to not centrifuge the EBs. The supernatant (retain 1 ml of media) was removed, 2 ml of DIM was added and then 500 μl of resuspended media was added in a drop-wise manner using 1 ml pipette tip such that the EBs were distributed uniformly across the plate.
iii. Gently the culture plate was shaken to aid the uniform distribution of the aggregates and the plate was carefully placed back in the incubator. It was ensured to not disturb the plate for 24 hours.

AggreWell™800 Microwell Culture Plates (Stem Cell Technologies) were used for EB formation for the same RPE differentiation method. Uniformity in terms of size of EBs generated using the multi-well plates was observed. However, no significant advantage was observed with respect to final RPE numbers, processing time or cost incurred. Therefore, ultra-low attachment dish was continued in use to form EBs. g. Day 7-8:
i. After 24 hours, the plates were observed for EB attachment. The majority of the EB's would attach and start exhibiting outgrowth from the periphery.

Floating EB's were discarded at this point while changing medium.
ii. A complete media change was given with 100% DIM (Refer Table-1 for volume).

### 3. Introducing Differentiation Propagation Media (DPM) for Rosettes Formation.

a. DPM was prepared as per the ingredients list provided in Table-3.

TABLE 3
Differentiation Propagation Media (DPM)

| | | | Final | |
|---|---|---|---|---|
| Components | For 500 ml | | concentration | |
| DMEM/F12 | 465 | ml | | |
| Fetal Bovine Serum | 10 | ml | 2% | |
| Sodium pyruvate (100 mM) | 5 | ml | 1 | mM |
| Sodium bicarbonate (7.5% | 5 | ml | 8.92 | mM |
| solution) | | | | |
| HEPES buffer (1M) | 5 | ml | 10 | mM |

| | | | |
|---|---|---|---|
| Non-essential amino acids (100X) | 5 | ml | 1X |
| N1 media supplement (100X) | 5 | ml | 1X |

N1 supplement was added to filtered media.

At this stage, media could be stored at 4° C. for 2 weeks.
b. Day 9: Gradually the medium was shifted from DIM to differentiation propagation media (DPM). One part of DIM media was replaced with DPM (1:1) (Example: For 1 ml media, 500 μl DIM and 500 μl DPM).
c. Complete media change (100%) was given with DPM.
d. Day 11-22: The cultures were fed with DPM on alternate days until day 22 (Refer Table-1 for volume)

At around day 20, rosette-like structures and epithelial clusters formations were observed indicating the onset of retinal differentiation (FIG. 1B).

### 4. Introducing Retinal Pigment Epithelium Maturation Media (RPEMM) for the Formation of Retinal Progenitor Cells

a. RPEMM was prepared as shown in Table-4.

TABLE 4
Retinal Pigment Epithelium Maturation Media (RPEMM)

| | | |
|---|---|---|
| | | Final |
| Components | For 500 ml | concentration |

| MEMα modified | 462 | ml | | |
|---|---|---|---|---|
| Fetal Bovine Serum | 25 | ml | 5% | |
| GlutaMAX (100X) | 5 | ml | 1X | |
| Taurine (50 mg/ml) | 2.5 | ml | 0.25 | mg/ml |
| Hydrocortisone (20 mg/ml) | 550 | μl | 10 | μg/ml |
| Tri-iodo-thyronine (2 mg/ml) | 15 | μl | 0.0065 | μg/ml |

| | | | |
|---|---|---|---|
| N1 media supplement (100X) | 5 | ml | 1X |

Growth factors and supplement were added to filtered media. The growth factors were reconstituted and stored as per manufacturer's instructions. (https://www.sigmaaldrich.com/IN/en/product/sigma/t0625; https://www.sigmaaldrich.com/IN/en/product/sigma/h6909; https://www.sigmaaldrich.com/IN/en/product/sigma/t5516)

The prepared media could be stored at 4° C. for 2 weeks.
b. Gradually the medium was switched from DPM to Retinal Pigment Epithelium Maturation Media (RPEMM).
c. Day 23: One part of DPM media was replaced with RPEMM (1:1) (Example: For 1 ml media, 500 μl DIM and 500 μl RPEMM).
d. Day 24: On day 24 there was switch to complete RPEMM (FIG. 1D).
e. Day 25-45: The cultures were fed with RPEMM on alternate days until day 40 (refer Table-1 for volume).

### 5. Day 40 (+5): Enrichment Step by Passaging of RPE

As shown in FIG. 1C, onset of pigmentation was observed in the cultures, indicating the fate commitment of retinal progenitor cells towards RPE cells. Further visible patches of pigmentation were observed in RPE culture plates (FIG. 1E). RPE cells could be further passaged between day 35 to day 45 after the onset of pigmentation for enriching the pigmenting cells and to obtain the matured RPE cells.
a. Matrigel coating: Matrigel coated plates were prepared as previously stated (Refer step 4 in Example 2).
b. For 1x6 well plate, 3 ml of gentle cell dissociation reagent (GCDR) and 3 ml StemPro Accutase (enzyme for dissociation) was aliquoted in a 15 ml tube and pre-warmed in incubator at 37° C. for 30 mins.
c. The culture medium was aspirated and gently washed once with CTS™ DPBS (1X) (-Ca²⁺/−Mg²⁺) 1 ml/well.
d. The cells were incubated with 1:1 ratio of pre-warmed GCDR and Accutase at 37° C. for 11-12 mins.

If required the cells were incubated for an additional 2 mins at Step d to ensure complete dissociation into single cells.
e. It was gently pipetted to dissociate into single cells or aggregates of 5 to 10 cells and diluted with RPEMM media.
f. The cell suspension was collected in a 15 ml tube and centrifuged at 200 g for 3 mins. The supernatant was removed.
g. The matrigel (extracellular matrix) was aspirated just before seeding cells and RPEMM media was added in each well (Refer Table-1 for volume).
h. The cell pellet was resuspended in RPEMM, and cells were seeded 1:2 or 1:3 in matrigel coated plates.
i. The cells were allowed to attach without disturbing for 24-48 hours.
j. After proper attachment of cells, media change was resumed.

### 6. Day 47-75 or Later

a. The cultures were fed with RPEMM on alternate days until day 75-80.

b. Day 60 (+5): Passaging RPE.

RPE cells can be passaged again around day 60 (+5) by following the steps mentioned in Enrichment step by passaging of RPE (Refer step as described above) and maintain up to day 90-100 by changing medium on alternate days. This step could be implemented for further scale-up if there was a need to generate larger numbers of RPE cells for pre-clinical safety and toxicological studies.

### 7. Day 75 or Later: Selection and Cryopreservation

RPE cells showed cuboidal monolayer with hexagonal morphology at day 75 with visible deposits of melanin pigment. Further, at day 120 there was an increase in pigmentation with hexagonal morphology as observed as shown in FIG. 1D. Cultures were frozen using commercially available cryoprotectant-CryoStorR) CS10 Freeze Media-BioLife Solutions in 1.8 ml NUNC cryotubes.
a. The cells were washed twice with CTS™ DPBS (1X) (-Ca²⁺/−Mg²⁺).
b. Pre-warmed GCDR and Accutase were added at 1:1 ratio and incubate for 10-12 mins at 37° C.

If enrichment step by passaging the RPE cells was not performed at day 40/45 then step c was followed, else directly step d was followed.
c. Manually the visibly non-pigmented cluster areas was removed if any (non-black) using 20-200 μl pipette tips and discarded. Gently the pigmenting patches were flushed out from the wells using 1 ml pipette tips. Flushing was repeated carefully in the presence of RPEMM until all the visibly pigmented patches have been successfully detached.
d. Cells were collected and mixed with equal volumes of RPEMM. Cells were centrifuged at 200 g for 2 mins at room temperature (20-25° C.) (FIG. 1F).

After counting the cells, the number of cryovials required to freeze approximately 1 million cells per vial in 1 ml of cold freezing medium was determined. Labelling was done accordingly with cell line name, cell type, initials, and date on each cryovial.
e. To the RPE cell pellet Cryostor CS10 freezing medium was added and the RPE cell suspension was then gently mixed.
f. The cells (in suspension) were cryopreserved in cryovials at 1 million cells per vial.
g. The cryovials were placed in Mr. Frosty freezing container and stored at −80° C. for 24 hours. After 24 hours, the cells were transferred to the liquid nitrogen storage tank and cryotank binder was updated.

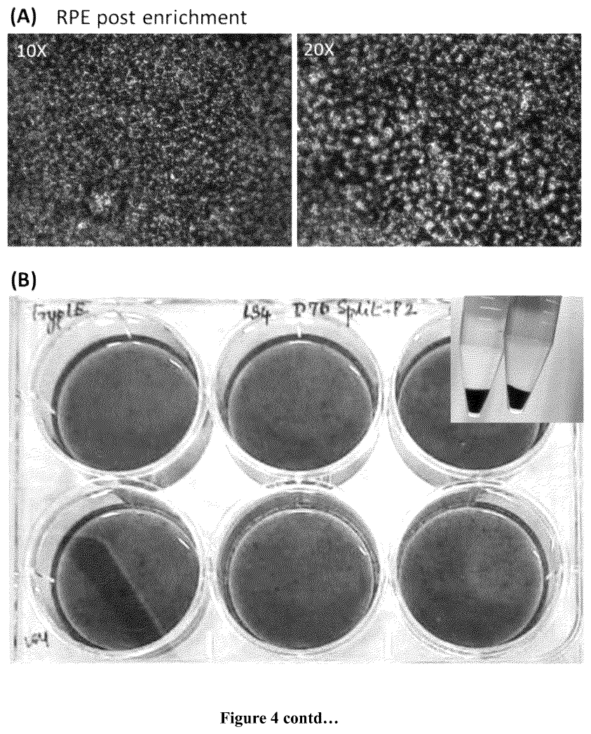

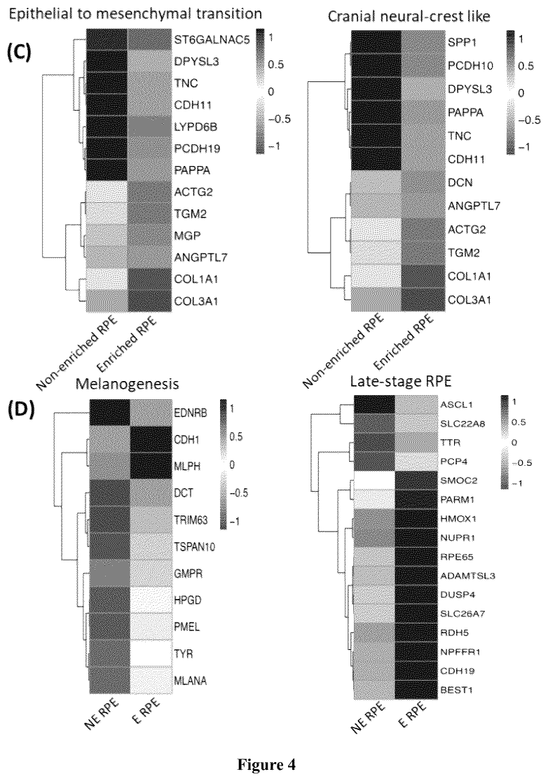

FIG. 4A depicts the phase contrast microscopic images of committed and mature RPE cells under low (10X) and high magnification (20×) showing heavy pigmentation and FIG. 4B depicts the pictures of the 6 well-plate with RPE cultures at late stage and 15 ml tubes containing RPE cells after centrifugation (inset) during the process of enrichment and scale-up.

### EXAMPLE 4

### Characterization of RPE In Vitro

### Protein Expression by Immunocytochemistry

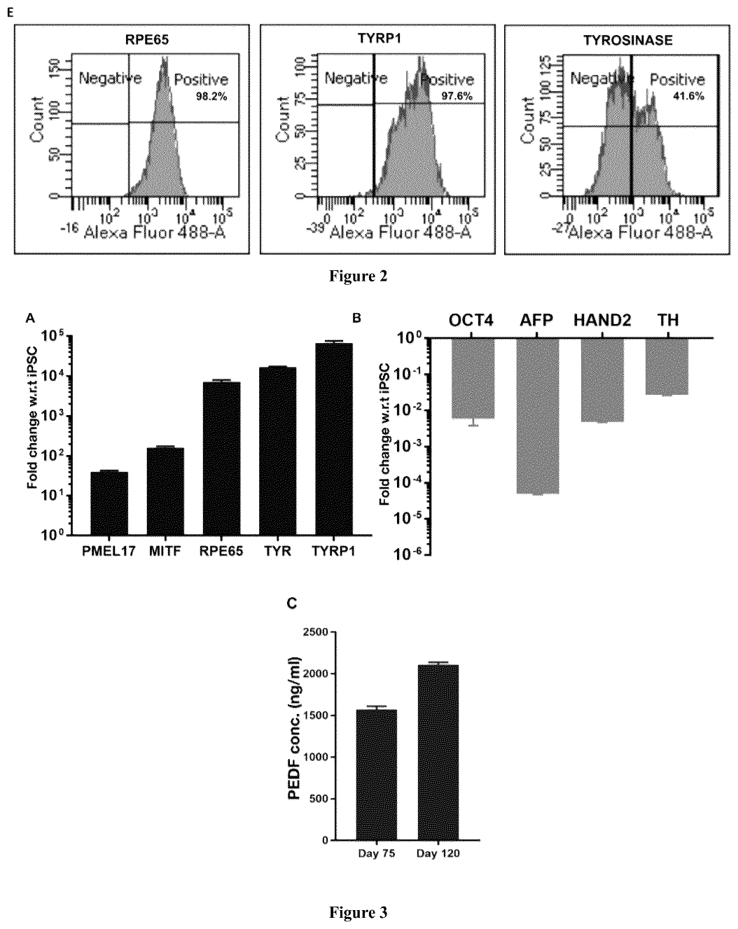

Immunocytochemistry of differentiation cultures was performed visualize the expression level and localization of stage-specific retinal markers and which, authenticated the successful differentiation of iPSC to RPE (FIG. 2A-D).

Timing: 4 hours-2 davs

TABLE 5
Immunocytochemistry (ICC) reagents

| | | |
|---|---|---|
| Reagents | Final Concentration | Amount (for 10 ml) |

Fixing solution

| | | | |
|---|---|---|---|
| Paraformaldehyde (PFA) | | 400 | mg |
| CTS-DPBS (1X) | 1X | 10 | ml |

(—Ca²⁺/Mg²⁺)

Blocking solution

| | | | |
|---|---|---|---|
| CTS-DPBS (1X) | 1X | 10 | ml |
| (—Ca²⁺/Mg²⁺) | | | |
| Triton-X-100 (10%) | 0.1% | 100 | μl |
| FBS | 4% | 400 | μl |

Antibody diluent

| | | | |
|---|---|---|---|
| CTS-DPBS (1X) | 1X | 10 | ml |
| (—Ca²⁺/Mg²⁺) | | | |
| FBS | 2% | 200 | μl |

Day 1
i. The culture medium was aspirated and gently washed thrice with CTS-DPBS (1X) (−Ca²⁺/−Mg²⁺) with fixing solution and incubated for 15-20 mins at room temperature (20-25° C.).
ii. The fixing solution was removed and gently washed thrice with CTS-DPBS (1X) (−Ca²⁺/−Mg²⁺).
iii. Permeabilization and blocking of cells was performed by adding blocking solution for 30 mins at room temperature (20-25° C.).
iv. Then the cells were gently washed thrice with CTS-DPBS (1X) (−Ca²⁺/−Mg²⁺).
v. Primary antibody diluted in antibody diluent was added to the cells and incubated overnight (16-20 hrs) at 4° C.

Day 2
vi. The primary antibody was removed and the cells were washed thrice with CTS-DPBS (1X) (−Ca²⁺/−Mg²⁺).
vii. Secondary antibody diluted in antibody diluent was added to the cells and then incubated in dark for 60 mins at room temperature (20-25° C.).
viii. The secondary antibody solution was removed and the cells were washed thrice with CTS-DPBS (1X) (−Ca²⁺/−Mg²⁺).
ix. The cells were counterstained with lug/ml concentration of DAPI and incubated in dark for 10 mins at room temperature (20-25° C.).
x. The DAPI solution was removed and the cells were washed once with CTS-DPBS (1X) (−Ca²⁺/−Mg²⁺).
xi. CTS-DPBS (1X) (−Ca²⁺/−Mg²⁺) (300 μl for 1 well of a 4 well plate) was added and the cells were visualized under a fluorescent microscope.

Fixed cells from step (ii) can be stored in CTS-DPBS (1X) (−Ca²⁺/−Mg²⁺) at 4° C. for 1 week before proceeding to staining.

### Observation

Immunostaining of iPSC derived RPE cultures was performed to check for marker expression during progressive days in retinal differentiation. The RPE commitment marker MITF (FIG. 2A); pigmentation specific proteins, such as Tyrosinase related protein TYRP1 (FIG. 2B) and melanocyte protein PMEL17 (FIG. 2C); and RPE maturation marker, RPE65 (FIG. 2D); showed higher expression in the RPE cells obtained by the method of the present disclosure.

### Immunophenotyping by Flow Cytometry

Flow cytometry analysis of RPE cells helps to quantify the levels of protein expression providing reconfirmation of successful differentiation of iPSC to RPE (FIG. 2E).

Timing: 3 hours

TABLE 6
FACS Buffer

| Reagents | Final Conc | Amount (for 10 ml) | |
|---|---|---|---|
| PBS | 1X | 10 | ml |
| FBS | 2% | 200 | μl |
| Triton-X-100 (10%) | 0.25% | 250 | μl |

Enzymatic dissociation of RPE into single cells
i. The culture media was aspirated and the cells were washed with CTS-DPBS (1X) (−Ca²⁺/−Mg²⁺).
ii. The cells were harvested enzymatically (GCDR and Accutase at 1:1 ratio for 10-12 mins; (Refer Section 7, steps a-d under Step-by-step method details) and gently pipetted to make a single cell suspension.
iii. The cells were centrifuged at 200 g for 3 mins and the supernatant was discarded.
iv. The pellet was washed by resuspending in CTS-DPBS (1X) (−Ca²⁺/−Mg²⁺) and centrifuged at 200 g for 3 mins.
v. The cells were fixed by adding 1 ml of 4% PFA for 10 mins at room temperature (20-25° C.).
vi. The cells were washed with CTS-DPBS (1X) (−Ca²⁺/−Mg²⁺) and centrifuged at 200 g for 3 mins and then CTS-DPBS (1X) (−Ca²⁺/−Mg²⁺) was completely removed.
vii. 1 ml of ice-cold methanol was added to the pellet and incubated at 4° C. for 20 mins.
viii. The cells were washed with FACS buffer (Refer Table-6) and centrifuged at 200 g for 3 mins.
ix. The cells were stained with primary antibody at an optimized concentration in 100 μl FACS buffer and incubated for 30 mins at room temperature (20-25° C.).
x. The wash was repeated with FACS buffer and centrifuged at 200 g for 3 mins.
xi. The secondary antibody in 100 μl FACS buffer was added to the pellet at optimized concentration and incubated in dark for 30 mins.
xii. The vials were vortexed every 10 mins to ensure mixing.
xiii. The cells were washed with CTS-DPBS (1X) (−Ca²⁺/−Mg²⁺) and resuspended in FACS buffer.
xiv. The cells were transferred to flow tubes for flow cytometry analysis.

Fixed cells from step (f) can be stored in CTS-DPBS (1X) (−Ca²⁺/−Mg²⁺) at 4° C. for 48-72 hours before proceeding to permeabilization and blocking.

Permeabilization, blocking, and immunolabelling were done simultaneously to avoid cell loss during washing steps.

### Observation

From FIG. 2E, it is evident that the RPE obtained by the differentiation of iPSCs were of high purity, as indicated by the high percentage of late stage RPE maturation markers like TYRP1, RPE65 and TYROSINASE in flow cytometry results.

### Gene Expression Analysis by Quantitative Real-Time PCR

Gene expression profiling helps to understand the molecular signature of these de novo generated RPE cells in comparison to undifferentiated iPSC.

Timing: 4-5 hours

### Purification of Total RNA

Pellet preparation:
i. The cells were collected in a sterile 1.5 ml tube (Refer Selection and Cryopreservation steps a-d in Example 3) and centrifuged for one min at 200 g to pellet down the cells.
ii. The supernatant was removed carefully and the pellet was snap frozen using liquid nitrogen. The snap-frozen cells were stored at −80° C. until RNA isolation.

RNA isolation:
iii. For RNA isolation, the RNeasy Mini kit (Qiagen) was used and all the steps were performed at room temperature (20-25° C.).
iv. 500 μl RLT buffer was added to the cell pellet and equal volume of 70% ethanol was added. The mixture was added into the spin column and swas subjected to a spin at 8000 g for 1 min.
v. The column was washed once with buffer RW1 and twice with buffer RPE. All the washing steps were performed at 8000 g for 1 min.
vi. For the final elution, 20 μl of RNAse free water was added directly to the spin column, incubated for a minute, and was subjected to a spin at 8000 g for 1 min to elute the RNA.
vii. The RNA was kept immediately on ice and quantified using a nanodrop.

cDNA synthesis:
viii. cDNA transcription was carried out using a Verso cDNA synthesis kit. All the reaction setup steps were performed on ice.
ix. Verso cDNA kit method recommends template RNA concentrations ranging from 1 μg to 1 μg. The cDNA synthesis reaction mix was performed on ice by using the volumes mentioned below.

TABLE 7
cDNA synthesis reaction

| Components | Volume |
|---|---|
| 5x cDNA synthesis buffer | 4 μl |
| dNTP mix | 2 μl |
| Oligo dT | 1 μl |
| RT enhancer | 1 μl |
| Verso enzyme mix | 1 μl |
| RNA | 1 μg |
| Nuclease free water | Up to 20 μl |

x. The RNA was incubated at 42° C. for 45 min for cDNA synthesis to occur and 95° C. for 2 min for enzyme inactivation. The synthesized cDNA can be used for qPCR analyses.

RT enhancer used from the kit degrades dsDNA during the transcription of RNA and it gets inactivated at 95° C. in the second step. This eliminates the need for DNase treatment.

### Observation

Gene expression profiles showed the purity of RPE cells post enrichment when compared with non-enriched population of RPE cells. The representative heat maps comprising of important non-RPE gene sets (epithelial to mesenchymal transition genes and cranial neural-crest like) showed downregulation in enriched RPE cells (FIG. 4C) and representative heat maps comprising of important RPE-specific gene sets (melanogenesis and late stage RPE) showed upregulation in enriched RPE cells (FIG. 4D).

### RT-qPCR

Analysing the expression of various genes by RT-qPCR.

RT-qPCRs were done using QuantStudio3 Real-Time PCR machine and the results were analyzed using the 2-ddCt method. The RT-qPCR experiment was performed preferably with minimal light.
xi. qPCR using SYBR green: 20 μl reaction mixture containing 10 μl of SYBR green mix (2x), 1 μl each of forward and reverse primer (stock 10 μM), 2 μl of cDNA (50 ng), and 6 μl of water was prepared.
xii. Run parameters of the qPCR reaction was as follows.

TABLE 8
qPCR cycle parameters- SYBR green

| Steps | Temperature | Time | | Cycles |
|---|---|---|---|---|
| UNG incubation | 50° C. | 2 | min | 1 |
| Polymerase | 95° C. | 2 | min | 1 |
| activation | | | | |
| Denaturation | 95° C. | 15 | s | 40 |
| Anneal/Extension | 60° C. | 1 | min | |

xiii. qPCR using TaqMan probes: A 20 μl reaction mixture containing 10 μl of TaqMan master mix (2x), 1 μl of the gene-specific probe (stock 20x), 2 μl of cDNA (50 ng), and 7 μl of water was prepared.
xiv. Run parameters of the qPCR reaction was as follows

TABLE 9
qPCR cycle parameters - TaqMan reagent

| Steps | Temperature | Time | | Cycles |
|---|---|---|---|---|
| UNG incubation | 50° C. | 2 | min | 1 |
| Polymerase | 95° C. | 30 | s | 1 |
| activation | | | | |
| Denaturation | 95° C. | 1 | min | 40 |
| Anneal/Extension | 60° C. | 30 | s | |

xv. Quantification of the gene expression: Here, the expression of test genes was normalized against the expression level of Beta-ACTIN/GAPDH as housekeeping genes. The fold change was calculated by normalizing the expression of the samples (RPE) against a control (iPSC).

### Observation

The gene expression analysis results for selected markers of RPE is depicted in FIG. 3. Gene expression analysis using qPCR for selected markers were represented as fold change compared to iPSC. All the positive markers, such as PMEL17, MITF, RPE 65, TYR, TYRP1 (FIG. 3A) showed higher expression and the negative markers, such as OCT4, alpha-fetoprotein (AFP), Heart and neural crest derivatives expressed 2 (HAND2), TH (FIG. 3B) showed a negligible expression in iPSC derived RPE of the present disclosure.

### Measurement of Extracellular Secretion of Protein by Enzyme-Linked Immunosorbent Assay (ELISA)

Secretion of Pigment-Epithelium Derived growth Factor (PEDF) is an important criterion to assess the polarity of in vitro generated RPE. Quantification of PEDF was done enzymatically using a commercially available kit (Human Serpin F1/PEDF Duoset ELISA kit).

Timing: 4-5 hours
i. The ELISA plate was coated with 100 μl of capture antibody and incubated overnight at room temperature (20-25° C.).
ii. The coated wells were washed with a wash buffer 3 times. All the steps were performed at room temperature (20-25° C.)
iii. 300 μl of reagent diluent was added as a blocking solution and the plate was incubated for 1 hr. The washing procedure 3 times with wash buffer was repeated.
iv. 100 μl of the sample (the day on which the cell supernatant was harvested) was added and standards diluted in reagent dilution to the coated wells were also added and the plate was incubated for 2 hours. Carefully, the plate was washed thrice with wash buffer.
v. 100 μl of detection antibody was diluted in reagent diluent and the plate was incubated for 2 mins.
vi. Carefully, the plate was washed thrice with wash buffer.
vii. 100 μl of Streptavidin-HRP was added to each well. The plate was covered and incubated for 20 min in dark.
viii. Carefully, the plate was washed thrice with wash buffer.
ix. 100 μl of substrate solution was added and incubated for 20 mins. It was ensured to avoid exposing the plates to direct light.
x. 50 μl of Stop Solution was added to each well and the plate was mixed gently.
xi. The optical density of each well was determined immediately at 450 nm and 540 nm for background subtraction using Varioskan LUX microplate reader.
xii. The concentration of PEDF from the samples was analyzed by plotting the standard values as a graph.

### Observation

The ELISA based quantification of secreted PEDF is depicted in FIG. 3C. ELISA based quantification of secreted Pigment Epithelium Derived growth factor (PEDF) from the in vitro culture supernatant at two different time points (day 75 and day 120) indicated an increase in polarity of the RPE obtained by the method of the present disclosure.

### EXAMPLE 5

### Preclinical Safety and Efficacy Studies in Animal Model

Sub-retinal transplantation of freeze-thawed RPE cells in animal model (rat) was performed to evaluate the efficacy and safety of the RPE cells in vivo, thereby providing important Investigational New Drug (IND) enabling pre-clinical data.
a. Animal maintenance

Timing: 2 months
i. Breed a colony of Pigmented RCS (RCS-p+/Lav) rats (NIH Rat Resource and Research Center, #315).
ii. Th animals were fed a standard laboratory chow and the animals maintained on a 12-hour dark/light cycle.
iii. The animals were weaned at postnatal day (PD) 21 and administered oral cyclosporine A (210 mg/L: Gengraf, North Chicago, IL) in drinking water. Animals were kept on cyclosporine A until sacrifice.
e. Cells preparation

The cells were prepared prior to injection under aseptic conditions following the disclosed protocol was extremely important for best performance of the cells post transplantation into animals.

Timing: 1 hour
a. The cells were removed from liquid nitrogen storage and thawed in a 37° C. water bath for 2 mins.
b. 1 ml of pre-warmed DMEM/F12 media was added dropwise to the vial.
c. The cells were transferred to a 15 ml tube containing 5 ml of pre-warmed DMEM/F12 media.
d. The cryotube was rinsed with 1 ml of DMEM/F12 media and added to 15 ml tube.
e. The cells were centrifuged at 150 g for 5 mins at room temperature (20-25° C.).
f. The supernatant was aspirated, and cells were re-suspended in 1 ml of pre-warmed DMEM/F12 media.
g. It was carefully pipetted up and down to re-suspend the cells.
h. 10 μl of cell suspension was removed and added to 10 μl of 0.08% Trypan blue solution.
i. After Pipetting up and down and cell suspension was loaded on to a hemacytometer (10 μl each side).
j. The viable cells were counted.
k. The cell suspension was centrifuged again at 150 g for 5 mins.
l. The cells were resuspended in appropriate volume to achieve desired concentration for injection.
   f. Subretinal Injections

Cryopreserved RPE frozen at day 75-80 were tested for subretinal injections and it was hypothesized that at this stage iPS-RPE were suitable for application in transplantation studies. Transplantation of cells at earlier stages of differentiation was possible but progenitor cell behaviour was not well known in vivo.

Timing: 1 day
a. One hour before injections, pupils were dilated with 1% tropicamide and 2.5% phenylephrine.
b. The eyes were anesthetized with topical 0.5% proparacaine HCl.
c. The animals were sedated with intraperitoneal ketamine/xylazine (100/10 mg/kg).
d. The corner of the eyelid was cut to allow for visualization of the posterior retina. A hemostat was used to reduce blood accumulation.
e. A small scleral/choroidal incision (˜1 mm) 2-mm posterior to the limbus was made in the dorsotemporal region using 27 gauge needle tip.
f. A small lateral corneal puncture was made using a 30-G needle to limit increases of intraocular pressure and reduce efflux of cells following injection.
g. Two microliters of cell suspension was delivered containing the total cell dose or control media into the subretinal space using a fine glass pipette (internal diameter, 75-150 μm) inserted into the subretinal space. The glass pipette was connected to a 10 μL Hamilton syringe (Hamilton, Reno, NV) with small-bore (400 μL total volume) microtubing.
h. After delivery, using a glass coverslip, the retina was observed under the microscope and the subretinal bleb was scored based on size and injection related issues, for example: bleeding, bubbles, cells in the vitreous.
i. 0.5% erythromycin ointment was placed on eyes and animals were let to recover from anesthesia.
j. On surgery day, dexamethasone (1.0 mg/kg) was administered intraperitoneally. Dexamethasone was administered to animals every other day for 2 weeks post-cell transplantation to minimize a potential inflammatory response.

It was ensured that injections should occur between PD22 and PD25. Animals need to be placed on cyclosporine A one day prior to injections.

g. Optokinetic Tracking (OKT)

Optokinetic thresholds should were assessed using a virtual optomotor system (VOS: CerebralMechanics) comprising of four computer monitors arranged in a square, displays facing inwards. On the monitors, a virtual cylinder displayed sine-wave gratings that was rotated either clockwise or counterclockwise permitting evaluation of both the left and right eyes independently: the left eye responds to clockwise, and the right eye to counterclockwise movement.

Timing: 1 day
a. The animal was placed on the pedestal in the center of the four computer monitors. The animals were allowed 5 mins to acclimate to the pedestal.
b. Using the Cerebral Mechanics software, the simple staircase examination was set up for both eyes, keeping the contrast set to 100%.
c. The program would begin with a low spatial frequency, the cylinder would rotate and if the grating was resolved, the animal would respond with a reflexive head and neck movement tracking the grating. If the animal responded then ‘yes’ was clicked, if the animal did not respond ‘no was clicked’. Based on this input, the spatial frequency of the grating would then incrementally increase until the animals no longer tracked the stimulus, resulting in a maximal spatial frequency threshold.
d. Once the examination was finished, the animal was taken off the pedestal and returned to their home cage.

The RCS rat has a near normal OKT through PD60. To determine the functional rescue resulting from the cell transplantation, this exam was most optimal no earlier than PD90.

### Observation

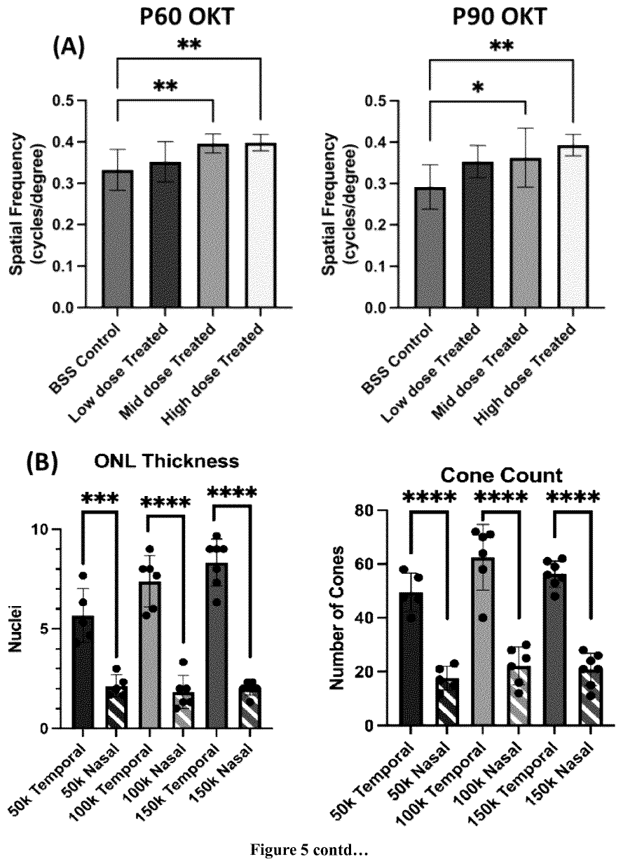

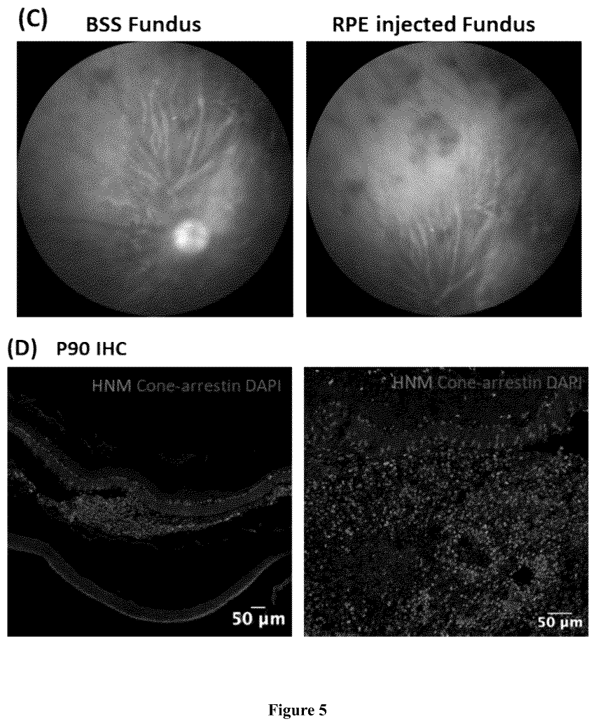

Behavioural analysis (functional test) in balanced salt solution (BSS, vehicle) and RPE injected RCS rats was measured by Optokinetic Threshold (OKT). The grating acuity of RCS rats decreased rapidly from birth to 4 months of age from 0.8 to 0.3 c/d. Visual acuity of cell treated eyes across all doses (low, middle, high) remained consistent over 70 days post injection (FIG. 5A).

e. Fundus Photography

A fundus camera or retinal camera was designed to observe the inner regions of eye, mainly the retina. Fundus images were obtained at any time post-injection to verify successful transplantation of cells and the length of cell survival.

Timing: 1 day
a. The pupils were dilated with 1% tropicamide and 2.5% phenylephrine.
b. The eyes were anesthetized with topical 0.5% proparacaine HCl.
c. The animals were sedated with intraperitoneal ketamine/xylazine (100/10 mg/kg).
d. The eyes were lubricated with 2.5% Goniovisc Hypromellose.
e. Using the Micron IV (Phoenix-Micron, Inc.) imaging system the white light was used to capture bright field images of the retina.
f. When imaging, the optic nerve head was used as a guide, but then the animal was positioned so that the peripheral/temporal region of the retina, where the injection took place, was in view.
g. 0.5% erythromycin ointment was placed on eyes and animals were let to recover from anesthesia.

### Observation

Fundus imaging of the saline (BSS) and RPE injected (sub-retinal) eye of RCS rats showed patches of pigmented (black) RPE cells in the cell injected group (FIG. 5C).

f. Tissue Preparation

Tissue samples were appropriately harvested and prepared for histology and immunohistochemistry studies. Sample preparation included processes, such as fixation, dehydration, embedding and sectioning.

Timing: 5 days
a. After euthanasia following IACUC guidelines, the eye was enucleated as rapidly as possible.
b. The enucleated eye was placed into cold 4% paraformaldehyde (PFA), while trying to maintain a 10-20x fixative to tissue ratio.
c. After 10-60 mins of fixation, the anterior chamber of the eye including the lens was removed and replaced the remaining eye cup into 4% PFA. The samples were placed in PFA on ice.
d. The eyes were placed in 2-8° C. for 24-48 hours.
e. After 24-48 hours, the PFA solution was removed and an equal volume of cold 10% sucrose solution in 1xPBS was added.
f. The eyes were placed in 2-8° C. for 24 hours.
g. The 10% sucrose solution was removed, and the solution was replaced with an equal volume of cold 20% sucrose solution.\
h. The eyes were placed in 2-8° C. for 24 hours. \
i. The 20% sucrose solution was removed and replaced with an equal volume of cold 30% sucrose solution.
j. The eyes were placed in 2-8° C. for 24 hours.
k. The eye cup was removed from the 30% sucrose solution and dab dried on a piece of filter paper.
l. The eye cup cut side was placed down on filter paper to allow the vitreous to drain out of the eye cup. This step was repeated until the filter paper remained dry.
m. The eye cup was placed into a labeled cryomold and filled with OCT media.
n. The eye cup was oriented toward the front of the cryomold, and the injection site was oriented at about 2′o clock for OS (oculus sinister left) eyes and 10′o clock for OD (oculus dextrus right) eyes.
o. The cryomold, containing the eye, was frozen in a liquid nitrogen bath. It was ensured that the liquid nitrogen does not directly touch the OCT media, but only contacts the walls of the cryomold (FIG. 3C) as direct contact of liquid nitrogen to OCT media created air bubbles, ruining sample integrity.
p. Once the media no longer had a glossy shine, it was removed from the liquid nitrogen bath.
q. The cryomold was tightly wrapped in an aluminum foil to try to limit the block's exposure to air.
r. The wrapped blocks were placed in a −20° C. freezer.
   g. Histology/Immunohistochemistry

Hematoxylin and eosin (H&E) and immunohistochemical staining are valuable tools for detecting histopathological changes and specific antigens in tissues.

Timing: 2-4 weeks
a. Eye blocks were sectioned at 12 μm.
b. Approximately 40 slides were obtained containing 4 sections per slide from each eye.
c. Sections were collected in a 5-slide series to provide a representative section every 60 μm on each slide throughout the eye-cup.
d. The first slide of each series was stained with cresyl violet or hematoxylin and eosin and the retinal damage/toxicity and evidence of photoreceptor rescue was examined.
b. For immunohistochemistry, the slides were dried for at least 30 mins after removing from freezer.
c. The slides were blocked for 45 mins with 4% horse serum, 1% bovine serum albumin (BSA), 0.5% triton x in phosphate buffered saline (PBS).
d. Primary antibodies (anti-cone arrestin, generously provided by W. Clay Smith, PhD, University of Florida) were added to blocking buffer and incubated overnight (minimum 15 hours) at 4° C.
e. The next day, the slides were washed 3x5 mins with 1xPBS.
f. Secondary antibodies (1:300) were added in blocking buffer and incubated for 45 mins in the dark at room temperature (20-25° C.).
g. The slides were washed 3x5 mins with 1xPBS.
h. The slides were incubated with DAPI in the dark for 10 min.
i. The slides were washed 2x5 min with 1xPBS.
j. Coverslips were placed on the slides with 100 μl of Fluoromount G.

### Observation

Quantification of retinal thickness and number of cones from low, middle and high dose RPE transplanted animals between nasal and temporal regions of the retina was calculated by immunostaining retinal sections with cone-arrestin. Data showed significant preservation of ONL in temporal region compared to nasal region (FIG. 5B).

h. Quantification of Photoreceptors

The outer nuclear layer thickness (ONL) was measured with the help of immunohistochemical staining as the primary indicator of photoreceptor rescue.

Timing: 1-2 weeks
a. A scanning laser confocal microscope (Leica SP5 using LAS AF software; Leica) was used to image stained slides. The laser intensity settings (gain) were kept consistent for each emission wavelength.
b. The Z-stack images were collected at ×10 and ×20 magnification at 1024×1024-resolution with 1-μm step size.
c. The color channels were kept separate as each color channel z-stack is flattened. Each color channel image was saved and the color merged image as TIFF files.
d. The ONL cell bodies were counted within the rows of the ONL in both temporal (injected) and nasal (non-injected) regions to obtain retinal thickness-nuclei values.
e. The cone arrestin positive cells were counted in both temporal (injected) and nasal (non-injected) regions to obtain cones per image values.
f. Three observers were blinded to the dosage and age group to generate counts.
g. Using data collected in steps 4, 5 and 6, average ONL counts and photoreceptor counts per group and present data from both temporal (injected) and nasal (non-injected) regions.

### Observation

Immunostaining (HNM) of retinal tissue sections showed survival of transplanted RPE cells in the subretinal space at P90 (Passage 90) with preserved ONL layer (cone-arrestin) and a good-looking retina indicating vision rescue.

Overall, the disclosed method provides an efficient protocol to obtain enriched mature RPE cells of high purity and significant therapeutic potential.

### Advantages of the Present Disclosure

The present disclosure provides a method for obtaining RPE cells from iPSCs, which exhibits the following advantages.
(a) This method generated mature, pigmented, and polarized RPE exhibiting functional characteristics such as secretion of cytokines-PEDF and VEGF from the apical and basal chambers.
(b) De novo generated RPE cells are mature, ciliated, and mimic the morphology, ultrastructure and molecular signature of native RPE; they are also suitable for in vivo cell transplantation studies.
(c) The method includes an enrichment step, making it useful for large-scale GMP manufacturing of pure RPE population.
(d) RPE cells produced following this method are appropriate for cell replacement therapy for macular degeneration.
(e) RPE cells produced following this method can benefit through its neuroprotective effects for other retinal diseases associated with early and late-stage photoreceptor degeneration.
(f) Owing to their similarity to native RPE tissue with respect to structure, molecular signature, and function, they are suitable for modelling diseases associated with retinal degeneration.

1. A method for obtaining RPE (retinal pigment epithelium) cells from iPSCs (induced pluripotent stem cells) comprising the steps of:
(a) generating embryoid bodies from a culture of iPSCs, wherein the embryoid bodies are in non-adherent suspension culture,
(b) plating the embryoid bodies on a culture dish coated with a suitable extracellular matrix in differentiation induction media (DIM) and culturing for 6-8 days to obtain neuroectoderm lineage, wherein the DIM comprises at least one WNT pathway inhibitor and at least two SMAD pathway inhibitors,
(c) culturing the neuroectoderm lineage for 11-22 days in differentiation propagation media (DPM) for rosette formation, wherein the DPM does not comprise any inhibitor,
(d) culturing the rosettes of step (c) in Retinal Pigment Epithelium Maturation Media (RPEMM) for 23-45 days to facilitate retinal progenitor cells formation, and
(e) plating the retinal progenitor cells of step (d) on a culture dish coated with suitable extracellular matrix and culturing for 47-75 days in RPEMM to obtain RPE cells.

2. The method as claimed in claim 1, wherein the method further comprises enriching the RPE cells, comprising:
a) enzymatically dissociating the RPE cells from the extracellular matrix to obtain a single cell suspension of RPEs; and
b) plating the single cell suspension of RPEs of step (a) on a culture dish coated with suitable extracellular matrix and culturing for 75-100 days in RPEMM.

3. The method as claimed in claim 1, wherein the culture of iPSCs has a confluency in the range of 80%-90%.

4. The method as claimed in claim 1, wherein generating the embryoid bodies in step (a) comprises:
(a) culturing the iPSCs for 24 hours in growth media to form embryoid bodies, wherein the growth media comprises an expansion media and a ROCK inhibitor, and
(b) gradually contacting the developing embryoid bodies from the growth media to the DIM by (i) culturing the developing embryoid bodies in a media composition comprising the expansion media and DIM in a ratio of 3:1 for 24 hours, (ii) culturing the developing embryoid bodies obtained in step (i) in a media composition comprising the expansion media and DIM in a ratio of 1:1 for 24 hours, and (iii) culturing the embryoid bodies obtained in step (ii) in DIM for 24 hours followed by plating the embryoid bodies on a culture dish.

5. The method as claimed in claim 1, wherein the rosette formation in step (c) comprises:
(i) maintaining the neuroectoderm lineage in DIM for 24 hours;
(ii) maintaining the neuroectoderm lineage obtained in step (i) in a media composition comprising DIM and DPM in a ratio of 1:1 for 24-48 hours; and
(iii) culturing the neuroectoderm lineage obtained from step (ii) in DPM for 24 hours for facilitating rosette formation.

6. The method as claimed in claim 1, wherein the retinal progenitor cells formation in step (d) comprises:
(i) maintaining the rosettes in DPM for 24 hours;
(j) maintaining the rosettes obtained in step (i) in a media composition comprising DPM and RPEMM in a ratio of 1:1 for 24 hours; and
(k) culturing the rosettes obtained from step (j) in RPEMM for 24 hours for facilitating the formation of retinal progenitor cells.

7. The method as claimed in claim 1, wherein the at least one suitable extracellular matrix is selected from matrigel, laminin, vitronectin, fibronectin, collagen, poly-L-lysine, poly-L-ornithine, or combinations thereof.

8. The method as claimed in claim 1, wherein the at least one WNT pathway inhibitor is selected from 4-(1,3,3a,4,7,7a-Hexahydro-1,3-dioxo-4,7-methano-2H-isoindol-2-yl)-N-8-quinolinyl-Benzamide, 5-(Phenylsulfonyl)-N-piperidin-4-yl-2 (trifluoromethyl)benzene sulfonamide, 2-(2′,3-Dimethyl-[2,4′-bipyridin]-5-yl)-N-(5-(pyrazin-2-yl) pyridin-2-yl) acetamide, 2-(4-(2-methyl pyridin-4-yl)phenyl)-N-(4-(pyridin-3-yl)phenyl) acetamide, 8-Tetrahydro-2-[4-(trifluoro methyl)phenyl]-4H-thio pyrano[4,3-d]pyrimidin-4-one, or combinations thereof.

9. The method as claimed in claim 1, wherein the at least two SMAD pathway inhibitors are selected from 4-[4-(1,3-benzodioxol-5-yl)-5-pyridin-2-yl-1H-imidazol-2-yl]benzamide, 4-(6-(4-(piperazin-1-yl)phenyl) pyrazolo[1,5-a]pyrimidin-3-yl) quinoline, 3-[(1R)-1-(2,6-dichloro-3-fluorophenyl) ethoxy]-5-(1-piperidin-4-ylpyrazol-4-yl) pyridin-2-amine, 5-chloro-2-N-[2-methoxy-4-[4-(4-methylpiperazin-1-yl) piperidin-1-yl]phenyl]-4-N-(2-propan-2-ylsulfonylphenyl) pyrimidine-2,4-diamine, 9-ethyl-6,6-dimethyl-8-(4-morpholin-4-ylpiperidin-1-yl)-11-oxo-5H-benzo[b]carbazole-3-carbonitrile, 5-chloro-2-N-(5-methyl-4-piperidin-4-yl-2-propan-2-yloxyphenyl)-4-N-(2-propan-2-ylsulfonylphenyl) pyrimidine-2,4-diamine, or combinations thereof.

10. The method as claimed in claim 4, wherein the ROCK inhibitor is (1R,4r)-4-((R)-1-aminoethyl)-N-(pyridin-4-yl) cyclohexanecarboxamide.

11. The method as claimed in claim 2, wherein the enzymatic dissociation of RPE cells in step (a) is carried out using an enzyme selected from the group consisting of Accutase, Tryple select, TrypLE, Gentle Cell Dissociation Reagent (GCDR), and Dispase.

12. A retinal pigment epithelium cell, or a population thereof, produced by the method as claimed in claim 1.

13. A pharmaceutical composition comprising the retinal pigment epithelium cell or population thereof as claimed in claim 12; and pharmaceutically acceptable carriers.

14. The pharmacetical composition as claimed in claim 13, for use in treatment of retinal degeneration disease.

15. The pharmaceutical composition as claimed in claim 14, wherein the retinal degeneration disease is selected from a group consisting of age-related macular degeneration, and retinal diseases associated with early and late-stage photoreceptor degeneration.

16. A method of treating a retinal degeneration disease in a subject comprising: administering the pharmaceutical composition as claimed in claim 13 to the subject.
